(19) United States
(12) Patent Application Publication
(10) Pub. No.: US 2025/0099285 A1
(43) Pub. Date: Mar. 27, 2025

(54) PROTECTOR FOR STENOSING TENOSYNOVITIS

(71) Applicant: MENG-CHUN WANG, Taichung (TW)

(72) Inventor: MENG-CHUN WANG, Taichung (TW)

(21) Appl. No.: 18/809,727

(22) Filed: Aug. 20, 2024

(30) Foreign Application Priority Data
Sep. 22, 2023 (TW) 112136192

(51) Int. Cl. A61F 5/37

(52) U.S. Cl. CPC A61F 5/3723

(57) ABSTRACT

A protector for stenosing tenosynovitis is provided, including: a shell member, including a plurality of ribs defining a plurality of through holes through the shell member in a thickness direction and a plurality of channels transverse to the plurality of through holes; and an lining, attached to an inner side of the shell member, the lining being softer than the shell member. As a result, it is lightweight, comfortable to wear, and has good structural strength and protection.

### BACKGROUND OF THE INVENTION

### Field of the Invention

The present invention relates to a protector, particularly to a protector for stenosing tenosynovitis.

### Description of the Prior Art

In daily life, many tasks are performed by hands, such as holding, pinching and gripping, which require frequent and extensive use of the fingers, making the hands prone to injury. For instance, stenosing tenosynovitis is a common injury to parents, characterized by inflammation of the tendon sheath of the hand which can result in pain and swelling of the thumb and wrist due to inflammation of the tendons and their sheaths near the thumb.

Conventional techniques include fabric sleeves designed to be worn on the thumb and wrist to provide fixation of the thumb and reduce the burden, thereby achieving appropriate relief and treatment. However, fabric sleeves lack stability and protection. Conventional techniques also include hard shell protectors; however, the hard shell protectors with good support and protection have poor flexibility, which can cause pain and discomfort, as well as skin abrasions, so that they are unsuitable for long-term wear.

The present invention is, therefore, arisen to obviate or at least mitigate the above-mentioned disadvantages.

### SUMMARY OF THE INVENTION

The main object of the present invention is to provide a protector for stenosing tenosynovitis which is lightweight, comfortable to wear, and has good structural strength and protection.

To achieve the above and other objects, a protector for stenosing tenosynovitis is provided, including: a shell member, including a plurality of ribs defining a plurality of through holes through the shell member in a thickness direction and a plurality of channels transverse to the plurality of through holes; and an lining, attached to an inner side of the shell member, the lining being softer than the shell member.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a perspective view of an exemplary embodiment of the present invention;

FIG. 2 is an exploded view of an exemplary embodiment of the present invention;

FIG. 3 is cross-sectional view taken along a longitudinal direction according to an exemplary embodiment of the present invention;

FIGS. 4 and 5 are partial enlarged views of FIG. 3;

FIG. 6 is a cross-sectional view taken along a transverse direction according to of an exemplary embodiment of the present invention; and

FIG. 7 is a drawing showing application of an exemplary embodiment of the present invention.

### DETAILED DESCRIPTION OF THE PREFERRED EMBODIMENTS

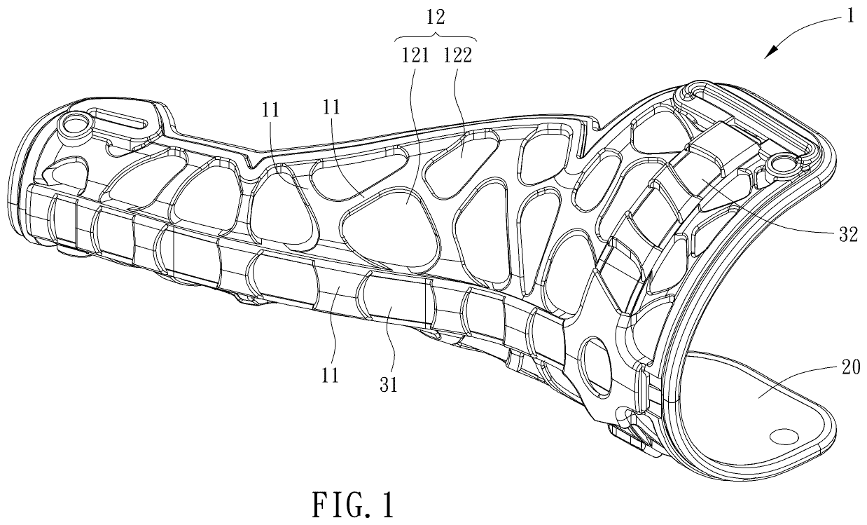

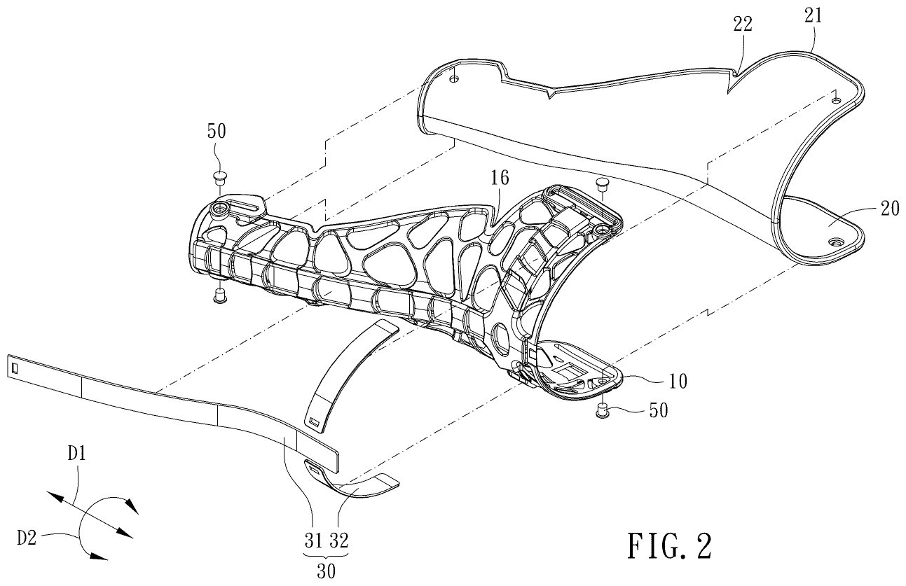

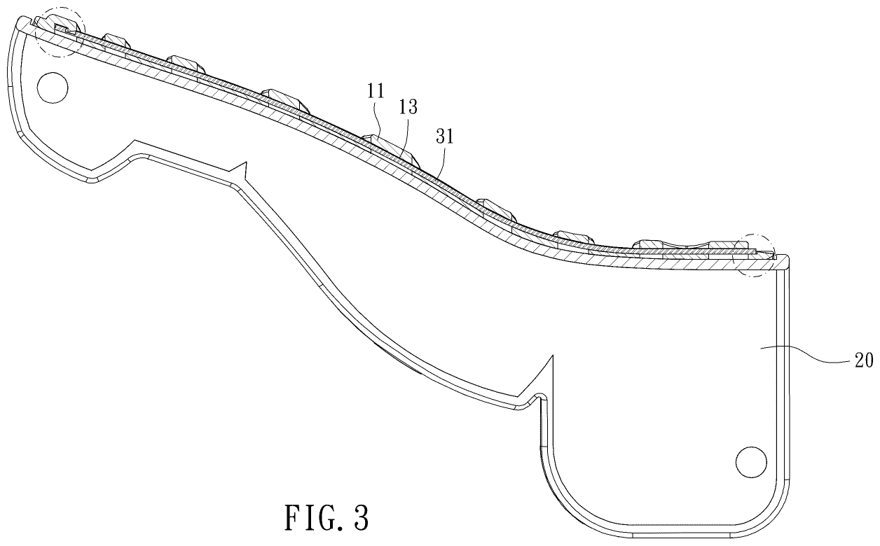

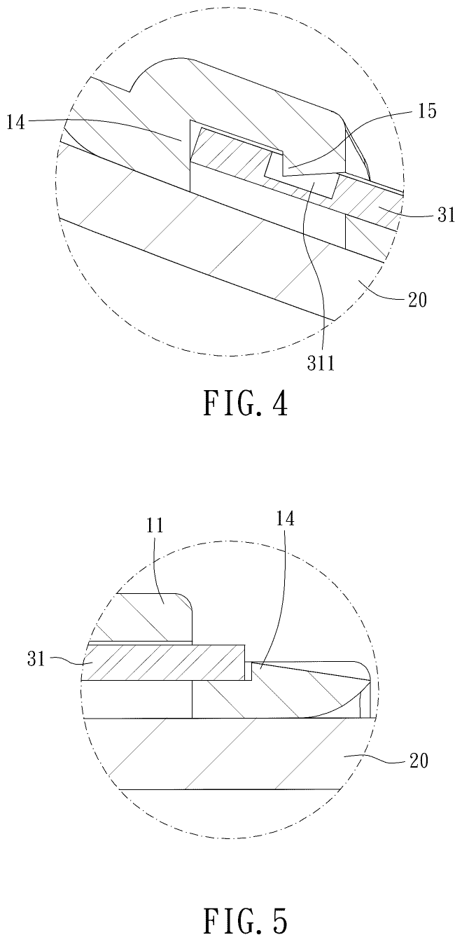

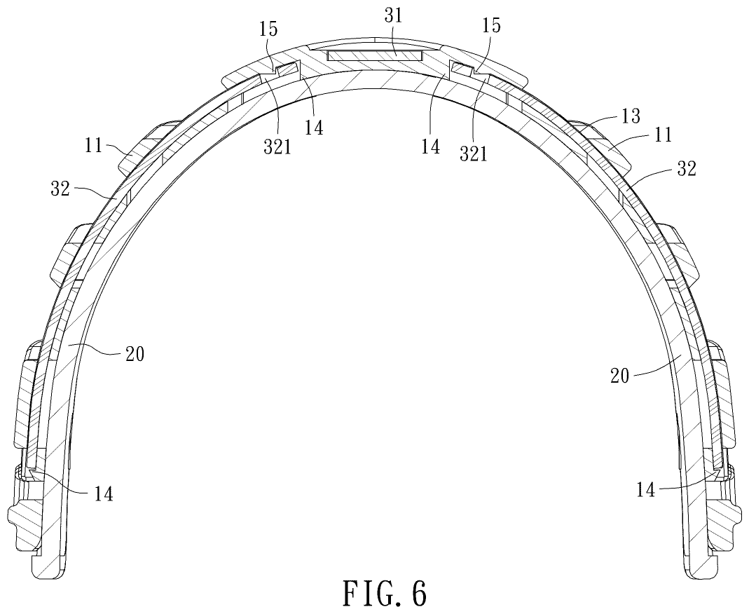

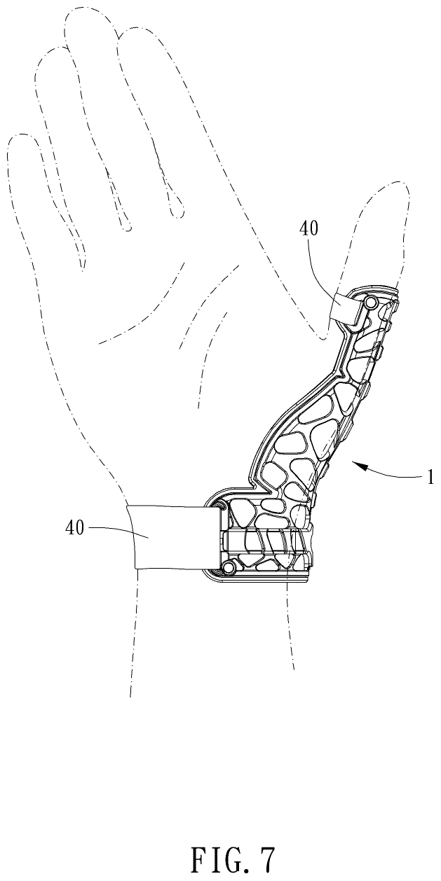

Please refer to FIGS. 1 to 7 for an exemplary embodiment of the present invention. A protector 1 for stenosing tenosynovitis of the present invention includes a shell member 10, a lining 20 and a reinforcing frame 30.

The shell member 10 includes a plurality of ribs 11, the plurality of ribs 11 define a plurality of through holes 12 in a thickness direction and a plurality of channels 13 transverse to the plurality of through holes 12, which is conducive to reducing weight and allowing appropriate flexing deformation, providing the necessary movement for the wearing regions. The lining 20 is attached to an inner side of the shell member 10, and the lining 20 is softer than the shell member 10. The shell member 10 which is relatively harder provides good support and fixation, while the lining 20 which is relatively softer offers comfortable wear. Optionally, the reinforcing frame 30 may be disposed through the plurality of channels 13 and located between the plurality of ribs 11, thereby increasing the structural strength and protection of the protector 1 for stenosing tenosynovitis.

In this embodiment, the shell member 10 defines a longitudinal direction D1 and a circumferential direction D2 transverse to the longitudinal direction D1. The plurality of channels 13 are arranged in the longitudinal direction D1 and in the circumferential direction D2. The reinforcing frame 30 includes at least one longitudinal rib 31 and at least one transverse rib 32 transverse to the at least one longitudinal rib 31. The at least one longitudinal rib 31 and the at least one transverse rib 32 are disposed through the plurality of channels 13 in the longitudinal direction D1 and in the circumferential direction D2, respectively. The shell member 10 further includes a plurality of blocking portions 14. The at least one longitudinal rib 31 and the at least one transverse rib 32 are blocked between the plurality of blocking portions 14. The shell member 10 further includes a plurality of hook portions 15. The at least one longitudinal rib 31 and the at least one transverse rib 32 each include a recess 311, 321 which may be blind holes or through holes. Each of the plurality of hook portions 15 is restrictedly engaged in the recess 311, 321 of one of the at least one longitudinal rib 31 and the at least one transverse rib 32, thus effectively and stably positioning and retaining the at least one longitudinal rib 31 and the at least one transverse rib 32. The at least one longitudinal rib 31 and the at least one transverse rib 32 may be separate components (for easy assembly and disassembly) or integrally formed of one piece (with greater structural strength). In other embodiments, the plurality of channels 13 may be arranged only in the longitudinal direction D1 or only in the circumferential direction D2.

In this embodiment, the reinforcing frame 30 includes one longitudinal rib 31 and two transverse ribs 32. The longitudinal rib 31 extends almost the entire length of the protector 1 for stenosing tenosynovitis. Each of the two transverse ribs 32 extends approximately halfway across the width of the protector 1 for stenosing tenosynovitis from each of opposing sides of one end of the longitudinal rib 31. Preferably, the protector 1 for stenosing tenosynovitis further includes at least one tightening strap 40. In this embodiment, the protector 1 for stenosing tenosynovitis includes two tightening straps 40. For example, when worn, the longitudinal rib 31 corresponds to an portion of a hand from the wrist to the thumb, the two transverse ribs 32 correspond to front, back and right sides of the wrist, and the two tightening straps 40 are configured to secure the wrist and thumb portions, and thus it effectively maintains the position of the protector 1 for stenosing tenosynovitis.

Preferably, two opposing sides of the shell member 10 each include at least one first notch 16 configured to correspond to at least one joint of a hand when worn; the lining 20 includes a peripheral flange 21, the lining 20 is arranged along and abutted against a periphery of the shell member 10, and two opposing sides of the lining 20 each include at least one second notch 22 corresponding to the at least one first notch 16. In this embodiment, the lining 20 includes a plurality of second notches 22 corresponding to the plurality of first notches 16, which provides good flexibility; the shell member 10 and the lining 20 are connected by a plurality of connection members 50 so that they can be fixed or detachable; the shell member 10 is preferably integrally formed of plastic or metal, which provides adequate support and protection; the lining 20 is made of woven fabric, non-woven fabric, rubber or silicone, which provides comfortable wear; the at least one longitudinal rib 31 and the at least one transverse rib 32 are made of metal (e.g., aluminum alloy) strips, offering better structural strength and protection; relative to the inner surface of the shell member 10, the plurality of channels 13 are located outside the plurality of through holes 12, which allows the at least one longitudinal rib 31 and the at least one transverse rib 32 to be easily and quickly assembled and disassembled.

Preferably, more than 50% of the plurality of ribs 11 extend non-parallel to each other, and a plurality of the plurality of through holes 12 are non-regular polygons, non-regular circles or non-regular ellipses, so that the shell member have strong and balanced structural strength in all directions. In this embodiment, the plurality of through holes 12 include a plurality of large holes 121 and a plurality of small holes 122, the small holes 122 are arranged adjacent to and along the periphery of the shell member 10, and the plurality of large holes 121 are located more internally in the shell member 10 relative to the plurality of small holes 122. The plurality of large holes 121 which are relatively larger help the protector 1 for stenosing tenosynovitis to fit more snugly on the wearing portion, and the plurality of small holes 122 which are relatively smaller allow the peripheral portion of the shell member 10 to have sufficient structural strength and support strength.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A protector for stenosing tenosynovitis including:
a shell member including a plurality of ribs, the plurality of ribs defining a plurality of through holes through the shell member in a thickness direction and a plurality of channels transverse to the plurality of through holes; and
an lining attached to an inner side of the shell member, the lining being softer than the shell member.

2. The protector for stenosing tenosynovitis of claim 1, further including a reinforcing frame, wherein the reinforcing frame is disposed through the plurality of channels and located between the plurality of ribs.

3. The protector for stenosing tenosynovitis of claim 2, wherein the shell member defines a longitudinal direction, the plurality of channels are arranged in the longitudinal direction, the reinforcing frame includes at least one longitudinal rib, and the at least one longitudinal rib is disposed through the plurality of channels in the longitudinal direction.

4. The protector for stenosing tenosynovitis of claim 3, wherein the shell member further includes a plurality of blocking portions, the at least one longitudinal rib is blocked between the plurality of blocking portions.

5. The protector for stenosing tenosynovitis of claim 2, wherein the shell member defines a circumferential direction, the plurality of channels are arranged in the circumferential direction, the reinforcing frame includes at least one transverse rib, and the at least one transverse rib is disposed through the plurality of channels in the circumferential direction.

6. The protector for stenosing tenosynovitis of claim 5, wherein the shell member further includes a plurality of blocking portions, and the at least one transverse rib is blocked between the plurality of blocking portions.

7. The protector for stenosing tenosynovitis of claim 2, wherein the shell member defines a longitudinal direction and a circumferential direction transverse to the longitudinal direction, the plurality of channels are arranged in the longitudinal direction and in the circumferential direction, the reinforcing frame includes at least one longitudinal rib and at least one transverse rib transverse to the at least one longitudinal rib, and the at least one longitudinal rib and the at least one transverse rib are disposed through the plurality of channels in the longitudinal direction and in the circumferential direction respectively.

8. The protector for stenosing tenosynovitis of claim 7, wherein the shell member further includes a plurality of blocking portions, the at least one longitudinal rib and the at least one transverse rib are blocked between the plurality of blocking portions.

9. The protector for stenosing tenosynovitis of claim 8, wherein the shell member further includes a plurality of hook portions, the at least one longitudinal rib and the at least one transverse rib each include a recess, and each of the plurality of hook portions is restrictedly engaged in the recess of one of the at least one longitudinal rib and the at least one transverse rib.

10. The protector for stenosing tenosynovitis of claim 1, wherein the lining includes a peripheral flange, and the peripheral flange is arranged along and abutted against a periphery of the shell member.

11. The protector for stenosing tenosynovitis of claim 1, wherein two opposing sides of the shell member each include at least one first notch configured to correspond to at least one joint of a hand when worn.

12. The protector for stenosing tenosynovitis of claim 11, wherein two opposing sides of the lining each include at least one second notch corresponding to the at least one first notch.

13. The protector for stenosing tenosynovitis of claim 9, wherein the lining includes a peripheral flange, the peripheral flange is arranged along and abutted against a periphery of the shell member; two opposing sides of the shell member each include a plurality of first notches configured to correspond to at least one joint of a hand when worn; two opposing sides of the lining each include a plurality of second notches corresponding to the plurality of first notches; the shell member and the lining are connected by a plurality of connection members; the shell member is integrally made of plastic or metal, the lining is made of woven fabric, non-woven fabric, rubber or silicone;
the at least one longitudinal rib and the at least one transverse rib are metal strips; more than 50% of the plurality of ribs extend non-parallel to each other; a plurality of the plurality of through holes are non-regular polygons, non-regular circles or non-regular ellipses;
relative to the inner surface of the shell member, and the plurality of channels are located outside the plurality of through holes; the protector for stenosing tenosynovitis further includes at least one tightening strap.

14. The protector for stenosing tenosynovitis of claim 1, wherein the plurality of through holes include a plurality of large holes and a plurality of small holes, the plurality of small holes are arranged adjacent to and along a periphery of the shell member, and the plurality of large holes are located more internally in the shell member relative to the plurality of small holes.
